(19) United States
(12) Patent Application Publication
(10) Pub. No.: US 2014/0073615 A1
(43) Pub. Date: Mar. 13, 2014

(54) Treatment of Psoriasis

(71) Applicants: Anders Carlsson, Stockholm (SE); Bengt Herslöf, Stockholm (SE); Jan Holmbäck, Vaxholm (SE)

(72) Inventors: Anders Carlsson, Stockholm (SE); Bengt Herslöf, Stockholm (SE); Jan Holmbäck, Vaxholm (SE)

(73) Assignee: LIPIDOR AB, Stockholm (SE)

(21) Appl. No.: 14/114,778

(22) PCT Filed: Apr. 30, 2012
(86) PCT No.: PCT/SE2012/000061

§ 371 Date: Oct. 30, 2013

(30) Foreign Application Priority Data
May 2, 2011 (SE) 1100340-7

(51) Int. Cl. A61K 47/24; A61K 31/593; A61K 31/573; A61K 31/575

(52) U.S. Cl. CPC A61K 47/24; A61K 31/575; A61K 31/593; A61K 31/573
USPC 514/167; 514/182

(57) ABSTRACT

A lipid layer forming composition for topical treatment of psoriasis comprises volatile silicone oil, polar lipid, C₂-C₄ aliphatic alcohol, and a pharmacologically effective amount of an agent for the treatment of psoriasis, wherein the silicone oil has a boiling point above 180° C., in particular above 200° C. The composition does not comprise polymer silicone. Also disclosed are corresponding methods of treatment and of manufacture of the composition.

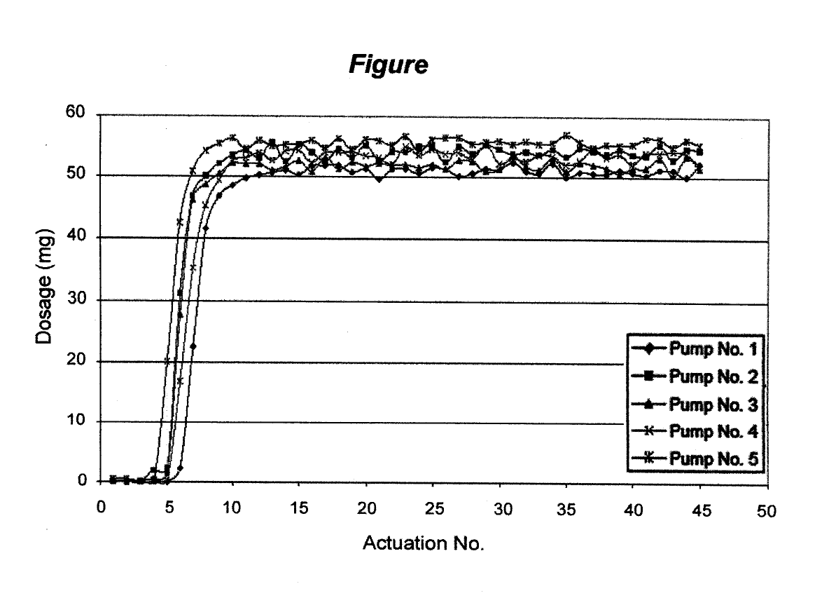

### FIELD OF THE INVENTION

The present invention relates to the treatment of psoriasis.

### BACKGROUND OF THE INVENTION

Psoriasis is a common chronic inflammatory skin disease. There is yet no cure for psoriasis. Mild to moderate psoriasis is treated topically by administration of topical corticosteroids and vitamin D₃ and analogues thereof, often in combination (E Vakirlis et al., Calcipotriol/betamethasone diproprionate in the treatment of psoriasis vulgaris. Ther Clin Risk Manag 4 (2008) 141-148 (http://www.ncbi.nlm.nih.gov/pmc/articles/PMC2503650/).

A problem with topical administration of agents pharmacologically effective in topical treatment of psoriasis is dosing. While overdosing should be avoided to keep adverse reactions at a minimum, underdosing jeopardizes adequate treatment. Since ointments and creams are difficult to apply evenly on the skin, the risk of wrong dosing is increased with such preparations.

### OBJECTS OF THE INVENTION

It is an object of the invention to provide a composition for treating psoriasis by topical administration of corticosteroids and vitamin D₃ and analogues thereof, including their combinations, which can be applied on the skin of a person suffering from the disease in a convenient and reproducible manner.

It is another object of the invention to provide a means for topical administration of the composition.

Still another object of the invention is to provide a corresponding method for treating psoriasis.

Further objects of the invention will be evident from the following summary of the invention, preferred embodiments thereof described in form of examples, as well as from the appended claims.

### SUMMARY OF THE INVENTION

According to the present invention is disclosed a lipid layer forming composition for topical treatment of psoriasis comprising volatile silicone oil, lipid, C₂-C₄ aliphatic alcohol, and a pharmacologically effective amount of an agent for the treatment of psoriasis, wherein the silicone oil has a boiling point above 180° C., in particular above 200° C. but not exceeding 300° C. Preferred active agents of the invention include 7-dehydrocholesterol (pro-vitamin D3), cholecalciferol (vitamin D3), calcipotriol hydrate, anhydrous calcipotriol, calcitriol, maxacalcitol, doxercalciferol, paricalcitol, inecalcitol, eldecalcitol, betamethanone and derivatives thereof, for instance betamethasone-17-valerate and betamethasone diproprionate. Preferred lipids of the invention are polar lipids, in particular membrane lipids selected from phospholipid, glycolipid, sphingolipid, and their mixtures. Polymer, non-volatile silicones are excluded from the composition of the invention.

The present invention is based on the insight that volatile silicone oils of a boiling point of 180° C. or higher, in particular of a boiling point of 200° C. or higher, can be used as an evaporating component of lipid carrier compositions for topical administration of an agent for treating psoriasis, the composition additionally comprising lipid and lower alcohol. “Evaporating component” indicates the capacity of silicone oils to evaporate, in spite of their high boiling point, within a short time upon application of the composition to the skin or other surface at ambient temperature or at a higher temperature. By their evaporation and the evaporation of the lower alcohol a layer of lipid is formed on the skin or other surface. The composition of the invention thus is capable of providing a stable coherent lipid layer on the skin of a person suffering from psoriasis. The composition of the invention is particularly suitable for administration by spraying onto the skin. Preferred means for its administration are devices for spray dosing known in the art, in particular those allowing precise dosing.

The present invention is further based on the finding that a particular class of solvents, volatile silicone oils, optionally in combination with a lower aliphatic alcohol, are particularly useful in formulating a carrier composition comprising lipid, into which an agent pharmacologically active in the treatment of psoriasis can be incorporated. After application onto the skin the composition of the invention forms an unstable lipid layer from which the volatile silicone oil and the lower aliphatic alcohol evaporate readily, leaving a stable residual layer substantially consisting of lipid and pharmacologically active agent. In respect of polar lipids the low viscosity of the composition of the invention seems to be due to their inability to form lyotropic liquid crystals, such as lamellar, hexagonal and various cubic phases of high viscosity. The lipid carrier composition and the pharmaceutical or cosmetic composition of the invention are clear and of low viscosity even at concentrations of polar lipid as high as 20% w/w.

In contrast, polar lipid compositions corresponding to those of the invention but in which the silicone oil component is substituted by a corresponding amount of water are slightly viscous dispersions at low polar lipid concentrations or thick gels at higher polar lipid concentration tested, for instance 20% by weight of the composition. The high viscosity of the latter composition does not allow administration by spraying. By using the volatile silicone oil as diluent instead of water, it is possible to incorporate a high amount of polar lipid while only insignificantly affecting viscosity.

Silicone oils of personal care grade or pharmaceutical grade useful in the invention are known in the art. Examples of useful silicone oils include dekamethyl-cyclopentasiloxane (Dow Corning® 245 Fluid and ST-Cyclomethicone 5-NF) and dodekamethylcyclohexasiloxane (Dow Corning® 246 Fluid). While pentasiloxanes and hexasiloxanes are preferred, hepta- and octasiloxanes are also potentially useful. The silicone oils can be cyclic siloxanes, that is, cyclomethicones, or linear siloxanes, that is, dimethicones. The silicone oils of the invention can be used in pure form or in admixture. While permethyl substitution is preferred, one or more methyl groups of a siloxane can be substituted by lower alkyl, in particular by ethyl, propyl or isopropyl. Siloxanes partially or fully substituted by lower trifluoroalkyl, in particular trifluoromethyl and pentafluoroethyl, are also useful in the invention.

In addition to chemical inertness the usefulness of the silicone oil of the invention is determined by its volatility. In spite of its high boiling point above 180° C., in particular above 200° C., the silicone oil of the invention evaporates readily due to the low heat of vaporization of this class of compounds. In the invention a silicone oil having a heat of vaporization (kJ/kg) at 25° C. of from about 100 kJ/kg to about 300 kJ/kg, more preferred of from about 120 kJ/kg to about 200 kJ/kg is particularly useful. Even more preferred is silicone oil having a heat of vaporization of from140 kJ/kg to about 180 kJ/kg at 25° C.

The silicone oil of the invention provides the composition of the invention with at least the following advantageous features: 1) the ability to incorporate high contents of polar lipid material; ii) the formation of thermodynamically stable solutions; iii) the low viscosity of the solutions formed making them suitable for administration by spraying.

The lower aliphatic alcohol of the invention is a C₂ to C₄ alcohol or a mixture of such alcohols. Preferred examples of alcohols are ethanol and 2-propanol. Other useful alcohols are glycerol and 1,2-propanediol.

The lipid of the invention is preferably a polar membrane lipid such as a phospholipid, a monoglyceride, a glycolipid, a sphingolipid or a mixture thereof. A particularly preferred phospholipid is phosphatidyl choline. Other preferred phospholipids are phosphatidyl ethanolamine and phosphatidyl inositol. A particularly preferred glycolipid is galactolipid. A preferred galactolipid is digalactosyl-1,2-diacylglycerol as such or in admixture with other galactolipids and/or phospholipids and/or sphingolipids.

The polar lipid of the invention can be described as lipids capable of interaction with water (as defined in D. Small, The Physical Chemistry of Lipids. Plenum Press 1986, section 4.3), for example formed of membrane lipid(s), that is, lipid constituents of biological membranes. Membrane lipids contain a polar, hydrophilic head group and one or more lipophilic hydrocarbon chains. This combination makes the membrane lipid molecules amphipathic and enables them to associate both with water and oil. Such membrane lipids can be classified according to their chemical structure, which is a function of how the polar head group is linked to the lipophilic chains. Sphingolipids (linked by sphingosine) and glycerolipids (linked by glycerol) are the two main groups. Depending on the characteristics of the polar head group sphingolipids and glycerolipids can be further classified as phospholipids comprising a phosphate ester head group and glycolipids comprising a carbohydrate head group. Depending on the specific nature of the carbohydrate group, membrane lipids are sometimes called, for instance, galactolipids, which are glycerolipids with galactose in the polar head group. Examples of common membrane lipids are phosphatidylcholine (PC), phosphatidylethanolamine (PE), and digalactosyldiacylglycerol (DGDG). Membrane lipids of interest can be extracted from, for example, egg yolk (egg lecithin), milk and dairy products, soybeans (soy lecithin), other oil crops oat kernels, and other cereal and grains. These extracts can be further treated to obtain, for instance, PC from soy beans and galactolipids from oats. Preferred polar lipids are galactolipids, in particular galactolipids from oat kernels, or phospholipids from soybeans (soy lecithin or soy-PC). Synthetic or semi-synthetic polar lipids and membrane lipid analogues based on a carbohydrate or phosphate ester moiety are also comprised by membrane lipids of the invention. Examples of synthetic polar lipids comprise dioleoylphosphatidylcholine and dioleoylphosphatidylethanolamine. Other lipids capable of interaction with water are monoglycerides, for example monooleylglycerol and esters of lower aliphatic alcohols and fatty acids, for example isopropyl myristate.

Technical scale commercial polar lipids useful in the invention can contain substantial amounts of non-polar lipids, so as to be composed of up to about 50% to 60% by weight of non-polar lipid. Thus, according to a further preferred aspect of the invention, the polar lipid component of the carrier composition or the pharmaceutical or cosmetic composition of the invention comprises a non-polar lipid in an amount of up to 30% by weight or more, such as up to 50% or 60% by weight and even up to 75% by weight. Non-polar lipids as components of polar lipids are preferably mono- and diglycerides and their mixtures, in particular monoglycerides. In a polar lipid of the invention a higher proportion of mono- and diglyceride, in particular of monoglyceride, can be tolerated than one of triglyceride.

The use of a lower aliphatic alcohol such as absolute ethanol for the dissolution of the lipid of the invention is particularly useful with a lipid of a low chain-melting temperature. The chain-melting temperature is the temperature at which the acyl chains of a lipid undergo a phase transition in an excess of water, from a solid-like state to a melted or liquid-like state. Membrane lipid materials like Lipoid S75, Lipoid S45, Phospholipon 50, Lipoid S100, and DOPC all have chain-melting temperatures below 0° C. and can thus be readily dissolved in C₂ to C₄ alcohol, in particular ethanol, at concentrations up to 50% by weight and even higher.

To manufacture the composition of the invention the lipid, in particular a polar lipid such as a membrane lipid, for instanced lecithin or fractionated oat oil, is dissolved in C₂ to C₄ alcohol and then diluted with volatile silicone oil. The pharmacologically active agent is preferably dissolved in the C₂ to C₄ alcohol or in a mixture of the alcohol and the lipid. The resulting composition is a low-viscous, sprayable, homogenous liquid. A typical example of such a composition is one consisting of the following excipients: 49% DC 345, 37% fractionated oat oil (LTP Lipid Technologies Provider AB, Sweden), and 14% by weight of absolute ethanol. The composition comprises, for instance, 1 mg of cholecalciferol per gram of total excipients. Fractionated oat oil is obtained from crude oat oil and is enriched in polar lipids. It typically contains about 50% by weight of non-polar lipid, such as triacylglycerol and diacylglycerol, and about 50% by weight of polar lipid, such as phospholipid and galactolipid. Typically, the content of digalactosyldiacylglycerol in a fractionated oat oil is about 20% by weight. Suitable fractionated oat oils are disclosed, for instance, in WO 99/44585 A1.

Lipids like phosphatidylethanolamine, for instance dioleylphosphatidyl-ethanolamine (DOPE), or sphingolipid, for instance sphingomyelin, can also be used as a polar lipid of the invention, optionally in admixture with other polar and/or non-polar lipids. DOPE has a chain-melting temperature of −16° C. in water and can be dissolved in absolute ethanol at 50% by weight or higher at elevated temperatures (>60° C.). Solutions of this kind can be diluted with volatile silicone oil such as DC 345, resulting in a clear liquid of low viscosity, for instance a viscosity lower than that of water.

The composition of the invention can further comprise antioxidant, for instance tocopherol and their derivatives such as (±)-a-tocopherol, ascorbic acid and their derivatives such as ascorbyl palmitate, butylated hydroxyanisole (BHA), butylated hydroxytoluene (BHT), fumaric acid, malic acid, propyl gallate, metabisulfates and their derivatives. The antioxidant can be present from about 0.0001% to about 5.0% w/w.

The composition of the invention can further comprise pharmaceutical excipients know in the art for improving its visual and/or sensitory acceptance, for instance fragrance agent such as menthol, and colorant.

Although small amounts of water, such as 1% or 2% and even up to about 5% by weight, can sometimes be tolerated, the lipid carrier composition of the invention is preferably substantially water-free, in particular has a water content of less than 1% or 0.1% by weight.

The pharmacologically active agent effective against psoriasis can be incorporated in the composition in an amount of from 0% to 2% by weight or even up to 5% by weight or more in respect of total non-volatile pharmaceutical excipients of the composition, in particular lipid, remaining upon evaporation of its volatile components. Because of the high efficiency of most anti-psoriasis agents their content on a weight basis will normally be less than 1%.

According to a preferred aspect of the invention the composition of the invention comprises or consists of from 5% by weight to 30% by weight of lipid, in particular phospholipid, from 5% by weight to 30% by weight of C₂ to C₄ alcohol, in particular ethanol, the remainder being volatile silicone oil, and agent pharmacologically active in the treatment of psoriasis, with the proviso that the content of volatile silicone oil is 50% by weight or more.

The composition of the invention can be applied to the skin by any suitable method, such as by spraying, dipping, brushing, dropping, rubbing in. Application by spraying is preferred.

According to the invention is thus disclosed a method of treating psoriasis, comprising: providing the composition of the invention in a container of a device for administration of the composition to diseased skin by spraying, the device allowing administration of measured doses of the composition; covering an area of diseased skin with the composition by spraying a desired number of doses on said area to form a layer of the composition thereon; allowing volatile components of the composition to evaporate from the skin to transform the initially formed layer into a residual layer substantially consisting of lipid, in particular polar lipid, and agent pharmacologically active in the treatment of psoriasis, so as to provide for absorption of the agent or a portion thereof through the skin. Efficient concentrations of active agent can be determined without undue experimentation, keeping in mind that the systemic absorption of the pharmacologically active agent for the treatment of psoriasis, such as calcipotriol and betamethasone, through normal skin is less than 1% (Vakirlis, supra). Useful concentrations of calcipotriol and betamethasone in the residual polar lipid layer are in the order of 50 μg/g and 0.5 mg/g, respectively.

According to the present invention is also disclosed a method of manufacture of the composition of the invention, the method comprising: providing an agent pharmacologically active in the treatment of psoriasis, lipid, in particular polar lipid, volatile silicone oil, and C₂ to C₄ alcohol; mixing the components in any suitable manner to form the composition, in particular by dissolving the agent in alcohol or in a mixture of alcohol and lipid, and mixing the alcoholic solution thus formed with lipid and silicone oil or silicone oil, respectively.

The invention will now be described in greater detail by reference to a number of Examples and a single Figure illustrating the dosing reproducibility for the composition of the invention obtainable by use of a state-of-the-art spray dosage pump.

### DESCRIPTION OF PREFERRED EMBODIMENTS

### Materials

TABLE 1
Silicone oils and lipids used in formulation experiments

| Short | | | |
|---|---|---|---|
| name | Supplier, trade name | Chemical name, CAS No. | Lot No. |
| DC 345 | Dow Corning ® 345 | Dekamethylcyclopentasiloxane, | 5627357 |
| | Fluid | 541-02-6 | |
| DC 245 | Dow Corning ® 245 | Dekamethylcyclopentasiloxane, | 5480964 |
| | Fluid | 541-02-6 | |
| DC 246 | Dow Corning ® 246 | Dodekamethylcyclohexasiloxane, | 5264620 |
| | Fluid | 540-97-6 | |
| 5-NF | Dow Corning ® ST- | Dekamethylcyclopentasiloxane, | |
| | Cyclomethicone 5-NF | 541-02-6 | |
| P-50 | Lipoid GmbH | Phospholipon ® 50, | |
| | | phosphatidylcholine | |
| IPNM | Evonik, Tegosoft M | Isopropyl myristate, 110-27-0 | |
| MCM | Aarhus Karlshamn | Akoline MCM, medium chain | 8192270 |
| | Sweden AB | monoglycerides | |
| DMPC | Lipoid GmbH, DMPC | Dimyristoyl phosphatidylcholine, | 562212-1/13 |
| | | 13699-48-4 | |
| DPPC | Lipoid GmbH, DPPC | Dipalmitoyl phosphatidylcholine, | 563086-1/94 |
| | | 2644-64-6 | |
| DOPC | Lipoid GmbH, DOPC | Dioleoyl phosphatidylcholine, | 566073-1/32 |
| | | 10015-85-7 | |
| DMPG | Lipoid GmbH, DMPG, | Dimyristoyl phosphatidylglycerol | 602081-1/10 |
| | Na salt | sodium salt, 200880-40-6 | |
| DPPG | Lipoid GmbH, DPPG, | Dipalmitoyl phosphatidylglycerol | 603032-1/36 |
| | Na salt | sodium salt, 200880-41-7 | |
| DMPE | Lipoid GmbH, DMPE | Dimyristoyl phosphatidyl- | 699201-1/05 |
| | | ethanolamine, 20255-95-2 | |
| DPPE | Lipoid GmbH, DPPE | Dipalmitoyl phosphatidyl- | 653004-1/19 |
| | | ethanolamine, 3026-45-7 | |
| DOPE | Lipoid GmbH, DOPE | Dioleoylphosphatidyl ethanolamine, | 656006-01/012 |
| | | 2462-63-7 | |
| CPL-GL | LTP, CPL ®- | Chromatographically purified | KGL06002 |
| | Galactolipid | galactolipids | |
| O65 | Swedish Oat Fiber, | Galactolipid enriched oat oil | PL 090219 |
| | Oatwell 65 oat oil | | |
| S45 | Lipoid GmbH, S45 | Soy bean lecithin, 8002-43-5 | 745303-1/926 |
| S75 | Lipoid GmbH, S75 | Soy bean lecithin, 8002-43-5 | 776132-07/918 |
| S100 | Lipoid GmbH, S100 | Soy bean lecithin, 8002-43-5 | 790551-7/910 |

### Compounds Pharmacologically Active in the Treatment of Psoriasis

7-Dehydrocholesterol (pro-vitamin D₃), Sigma-Aldrich 30800, lot 1431250V, 98,0%; cholecalciferol (vitamin D₃), Sigma-Aldrich C9756, lot 050M1877V, 99%; calcipotriol hydrate, Sigma-Aldrich C4369, lot 078K47194, 99,3%; calcitriol; tacalcitol; betamethasone-17-valerate, Sigma-Aldrich B0515, lot 077K1400, 98,1%; betamethasone dipropionate; momethasone furoate.

### EXAMPLE 1

Compositions of the invention. Five compositions of the inventions were prepared (Table 2) by dissolving the respective active substance or combination of active substances (Composition No. 5) in absolute ethanol. The alcoholic solutions were then mixed with the other constituents, polar lipid and silicone oil. The compositions thus obtained were colourless liquids of low viscosity.

TABLE 2
Compositions of the invention

| | | Active | |
|---|---|---|---|
| | | substance, mg | |
| | | per g composi- | Pharmaceutical |
| No. | Active substance | tion | constituents (w/w) |
| 1 | 7- | 1.2 | 15.7% Phospholipon 50, |
| | Dehydrocholesterol | | 15.7% ethanol abs., |
| | | | 68.7% DC 345 |
| 2 | Cholecalciferol | 1.0 | 10.0% Phospholipon 50, |
| | | | 10.0% ethanol abs., |
| | | | 80.0% DC 345 |
| 3 | Cholecalciferol | 1.0 | 10.0% Akoline MCM, |
| | | | 10.0%, ethanol abs., |
| | | | 80.0% DC 345 |
| 4 | Calcipotriol hydrate | 1.0 | 10.0% Phospholipon 50, |
| | | | 10.0% ethanol abs., |
| | | | 80.0% DC 345 |
| 5 | Calcipotriol hydrate + | 1.1 + 2.1 | 10.0% Phospholipon 50, |
| | betamethasone-17- | | 10.0% ethanol abs., |
| | valerate | | 80.0% DC 345 |
| 6 | Calcipotriol anhydrous | 0.05 | 19.0% Phospholipon 50, |
| | | | 20.0% ethanol abs., |
| | | | 60.0% 5-NF |
| 7 | 7- | 10 | 19.0% Phospholipon 50, |
| | Dehydrocholesterol | | 20.0% ethanol abs., |
| | | | 60.0% 5-NF |
| 8 | 7- | 10 | 19.0% DPMC, |
| | Dehydrocholesterol | | 20.0% ethanol abs., |
| | | | 60.0% 5-NF |
| 9 | 7- | 10 | 19.0% MCM, |
| | Dehydrocholesterol | | 20.0% ethanol abs., |
| | | | 60.0% 5-NF |
| 10 | 7- | 10 | 19.0% IPM, |
| | Dehydrocholesterol | | 20.0% ethanol abs., |
| | | | 60.0% 5-NF |

The concentration of calcipotriol in composition no. 6 was monitored by HPLC. No degradation was detected in samples stored protected from light at room temperature or at 40° C. for one week.

### EXAMPLE 2

Compositions of the invention. Nine compositions of the invention were prepared (Table 3) by dissolving calcipotriol anhydrous and sodium oleate or acetic acid in absolute ethanol. The alcoholic solutions were then mixed with polar lipids and silicone oil with addition of antioxidant. The compositions thus obtained were colourless to yellowish liquids of low viscosity.

TABLE 3
Compositions (w/w) of the invention.

| Comp. | S-100 | MCM | Calcipotriol | Vitamin E | Sodium oleate | Acetic acid | Ethanol | 5-NF |
|---|---|---|---|---|---|---|---|---|
| 19a-1 | 3.1 | 2.9 | 0.0045 | 0.0012 | — | 0.039 | 14.8 | 79.2 |
| 19a-2 | 10.0 | 3.1 | 0.0051 | 0.0010 | 0.040 | — | 14.9 | 72.0 |
| 9a-3 | 3.0 | 10.2 | 0.0045 | 0.0010 | 0.042 | — | 15.0 | 71.8 |
| 19a-4 | 10.0 | 10.1 | 0.0051 | 0.0009 | — | 0.040 | 14.9 | 64.9 |
| 19a-5 | 3.1 | 3.0 | 0.0046 | 0.0037 | 0.039 | — | 15.0 | 78.9 |
| 19a-6 | 9.8 | 3.1 | 0.0052 | 0.0042 | — | 0.039 | 14.9 | 72.2 |
| 19a-7 | 3.1 | 10.2 | 0.0047 | 0.0041 | — | 0.039 | 14.9 | 71.8 |
| 19a-8 | 10.0 | 10.0 | 0.0041 | 0.0037 | 0.041 | — | 14.9 | 64.9 |
| 19a-9 | 6.4 | 6.4 | 0.0046 | 0.0022 | 0.020 | 0.021 | 15.0 | 72.1 |

### EXAMPLE 3

Compositions of the invention comprising isopropyl myristate. Nine compositions of the invention were prepared (Table 4) by dissolving calcipotriol anhydrous in absolute ethanol. The alcoholic solutions were then mixed with isopropyl myristate and silicone oil with addition of antioxidant. The compositions thus obtained were colourless liquids of low viscosity.

TABLE 4
Compositions of the invention comprising isopropyl myristate.

| Composition | IPM | Calcipotriol | Vitamin E | BHT | Ethanol | 5-NF |
|---|---|---|---|---|---|---|
| 19a-10 | 5 | 0.005 | 0 | 0.001 | 1 | 94 |
| 19a-11 | 20 | 0.005 | 0 | 0.001 | 20 | 60 |
| 19a-12 | 5 | 0.005 | 0.001 | 0 | 20 | 75 |
| 19a-13 | 20 | 0.005 | 0.001 | 0 | 1 | 79 |
| 19a-14 | 5 | 0.005 | 0 | 0.004 | 20 | 75 |
| 19a-15 | 20 | 0.005 | 0 | 0.004 | 1 | 79 |
| 19a-16 | 5 | 0.005 | 0.004 | 0 | 1 | 94 |
| 19a-17 | 20 | 0.005 | 0.004 | 0 | 20 | 60 |
| 19a-18 | 12.5 | 0.005 | 0.0025 | 0.0025 | 10.5 | 77 |

### EXAMPLE 4

Dosing reproducibility. Administration of composition No. 2 (Example 1) provided by the applicant was tested by the pump manufacturer Aero Pump GmbH, Hochheim/Main, Germany on five spray dosing pumps model AP3 Santos (graph, Figure). As shown in the graph inter-pump dosage reproducibility was excellent: set dosage 50 mg, observed dosage in the range of 50 mg to 56 mg. Intra-pump dosage relative standard deviation was in the order of 5%.

### EXAMPLE 5

Method of treatment. The composition of the invention (Example 1, composition No. 5) was compared with an commercially available ointment comprising same amounts of the same/corresponding active substances (Daivobet®, LEO Pharma, Sweden; contains betamethasone dipropionate instead of betamethasone valerate) by applying a corresponding amount to measured areas of the left (composition of the invention) and right (commercial composition) forearm of a male person (57 yrs) suffering from moderate psoriasis for five days. On a visual basis and in the opinion of the person the effect of the compositions was comparable. The person however noted that, from his standpoint, the composition of the invention was preferable by reason of its easy and non-irritating manner of application.

### EXAMPLE 6

Preparation of pharmaceutical compositions according to the invention. Compositions comprising the following agents pharmacologically active in the treatment of psoriasis were prepared:
(a) 7-Dehydrocholesterol (pro-vitamin D3), Sigma-Aldrich 30800, lot 1431250V, 98,0%;
(b) Cholecalciferol (vitamin D3), Sigma-Aldrich C9756, lot 050M1877V, 99%;
(c) Calcipotriol hydrate, Sigma-Aldrich C4369, lot 078K47194, 99,3%
(e) Betametasone-17-valerate, Sigma-Aldrich B0515, lot 077K1400, 98,1%. Lipids: Phospholipon 50, from soy beans (Lipoid Gmbh), containing about 57% by weight of phosphatidylcholine; Akoline MCM (AarhusKarlshamn Sweden AB). Cyclomethicone: DC 345 (Dow Corning).

The compositions were prepared using a stock lipid solution in absolute ethanol, in which the pharmacologically active agents were dissolved. The alcoholic solution was then mixed with the appropriate volume of Cyclomethicone. The compositions are listed in Table 5:

TABLE 5
Pharmaceutical compositions according to the invention

| Composition | | Pharmacologically | Content of | |
|---|---|---|---|---|
| Number | Charge no. | active agent | active agent | Vehicle |
| 1 | ACA110217 | 7-dehydrocholesterol | 1.2 mg/g | 15.7% Phospholipon 50, |
| | | | | 15.7% absolute ethanol, |
| | | | | 68.7% DC 345 |
| 2 | ACA110223-2 | cholecalciferol | 1.0 mg/g | 10.0% Phospholipon 50, |
| | | | | 10.0% absolute ethanol, |
| | | | | 80.0% DC 345 |
| 3 | ACA110223-3 | cholecalciferol | 1.0 mg/g | 10.0% Akoline MCM, |
| | | | | 10.0% absolute ethanol, |
| | | | | 80.0% DC 345 |
| 4 | ACA110304-1 | calcipotriol hydrate | 1.0 mg/g | 10.0% Phospholipon 50, |
| | | | | 10.0% absolute ethanol, |
| | | | | 80.0% DC 345 |
| 5 | ACA110328-1 | calcipotriol hydrate | 1.1 mg/g | 10.0% Phospholipon 50, |
| | | betametasone-17-valerate | 2.1 mg/g | 10.0% absolute ethanol, |
| | | | | 80.0% DC 345 |

The compositions of Table 5 are physically stable liquids of low viscosity. Composition no. 5 is an example of a combination of an agent pharmacologiocally active against psoriasis with an anti-inflammatory agent, betamethasone-17-valerate. This composition can be compared with the known preparation Daivobet® salva (LEO Pharma, Sweden) containing 0.050 mg/g of calcipotriol (as the hydrate) and 0.5 mg/g betametasone (as the dipropionate). The concentrations of the active agents are far from those maximally obtainable. Neither have the concentrations of the components of the vehicle been optimized. Cyclomethicone can, for instance, be substituted by another suitable volatile silicone oil. The selected lipids can be substituted in part or fully by other suitable polar and non-polar lipids, depending on the desired properties of the film remaining on the skin after evaporation of the volatile silicone oil.

### EXAMPLE 6

Test of the pharmaceutical carrier of the invention on patients with mild/moderate psoriasis. The test subjects had been on a long-term treatment schedule with the anti-psorisasis state-of-the-art cream composition Daivonex®. Thirty-one persons of both genders (17 m, 15 f; age from less than 20 years up to more than 80 years) regularly treated with Daivonex ® participated in the study. They were informed that they would receive the carrier composition of the invention (consisting of 60% w/w of Cyclomethicone 5-NF, 20% w/w of Phospolipon 50, and 20% w/w of absolute ethanol) administered by spraying on the skin (model AP3 Santos spray dosing pump; Aero Pump GmbH, Hochheim/Main, Germany). After administration the participants were given a questionnaire providing background information about the test and asked to not their preference on the questionnaire. Twenty-three participants preferred the composition of the invention, five preferred the Daivonex® cream, and the remainder considered both compositions equal or did not give an answer. The result is statistically significant (p<0.01) in favour of the composition of the invention carrier.

1. Lipid layer forming composition for topical treatment of psoriasis comprising volatile silicone oil, lipid, C₂-C₄ aliphatic alcohol, and a pharmacologically effective amount of an agent for the treatment of psoriasis, wherein the silicone oil has a boiling point above 180° C., with the proviso that the composition does not comprise polymer silicone.

2. The composition of claim 1, wherein the silicone oil has a heat of vaporization (kJ/kg) at 25° C. of from about 100 kJ/kg to about 300 kJ/kg.

3. The composition of claim 1, consisting essentially of volatile silicone oil, polar lipid, C₂-C₄ aliphatic alcohol, and an agent for treatment of psoriasis.

4. The composition of claim 1, wherein the volatile silicone oil comprises one of dekamethylcyclopentasiloxane and dodekamethyl-cyclohexasiloxane.

5. The composition of claim 1, wherein the volatile silicone oil comprises one of heptasiloxane and octasiloxane.

6. The composition of claim 1, wherein the lipid comprises a membrane lipid selected from phospholipid, glycolipid, sphingolipid, and their mixtures.

7. The composition of claim 1, wherein the C₂-C₄ aliphatic alcohol is selected from the group consisting of ethanol, 2-propanol, 1,2-propanediol, glycerol, and their mixtures.

8. The composition of claim 1, comprising less than 1% by weight of water.

9. The composition of claim 1, comprising of from 5% by weight to 30% by weight of lipid, from 5% by weight to 30% by weight of C₂ to C₄ alcohol, the remainder being volatile silicone oil, and an agent pharmacologically active in the treatment of psoriasis, with the proviso that the content of volatile silicone oil is 50% by weight or more.

10. The method of claim 1, wherein the pharmacologically active agent is selected from corticosteroid, vitamin D₃ and vitamin D₃ analogue.

11. The method of claim 10, wherein the pharmacologically active agent is any of: 7-dehydrocholesterol (pro-vitamin D₃), cholecalciferol (vitamin D₃), calcipotriol hydrate, calcitriol, tacalcitol, betamethasone-17-valerate, betamethasone diproprionate, and momethasone furoate.

12. A method of treating psoriasis, comprising:
providing the composition of claim 1 in a container of a device for administration to diseased skin by spraying, the device allowing administration of measured doses of the composition;
covering an area of diseased skin with the composition by spraying a desired number of doses on said area to form a layer of the composition thereon; and
allowing volatile components of the composition to evaporate from the skin to transform the initially formed layer into a residual layer consisting essentially of lipid and pharmacologically active agent, so as to provide for absorption of the agent or a portion thereof through the skin.

13. The method of claim 12, wherein the temperature is from about 20° C. to about 45° C.

14. The method of claim 12, wherein the applied amount of composition is selected so as to obtain a stable lipid layer of from 1 μm to 500 μm thickness.

15. A method of manufacture of the composition of claim 1, comprising:
providing an agent pharmacologically active in the treatment of psoriasis, lipid, volatile silicone oil, and C₂ to C₄ alcohol; and
mixing the components to form the composition.

16. The composition of claim 1, wherein the lipid is a polar lipid.

17. The composition of claim 1, wherein the silicone oil has a boiling point above 200° C.

18. The composition of claim 9 wherein the lipid is phospholipid and the C₂ to C₄ alcohol is ethanol.

19. The composition of claim 1, wherein the silicone oil has a heat of vaporization (kJ/kg) at 25° C. of from 120 kJ/kg to 200 kJ/kg.

20. The composition of claim 1, wherein the silicone oil has a heat of vaporization (kJ/kg) at 25° C. of from 140 kJ/kg to 180 kJ/kg.
